(19) United States
(12) Patent Application Publication
(10) Pub. No.: US 2014/0135634 A1
(43) Pub. Date: May 15, 2014

(54) NONINVASIVE METHOD AND APPARATUS TO MEASURE CENTRAL BLOOD PRESSURE USING EXTRINSIC PERTURBATION

(71) Applicants: Osvaldas Pranevicius, New York, NY (US); Mindaugas Pranevicius, Forest Hills, NY (US); Henrikas Pranevicius, Kaunas (LT)

(72) Inventors: Osvaldas Pranevicius, New York, NY (US); Mindaugas Pranevicius, Forest Hills, NY (US); Henrikas Pranevicius, Kaunas (LT)

(21) Appl. No.: 13/676,277

(22) Filed: Nov. 14, 2012

(51) Int. Cl. A61B 5/022; A61B 5/00; A61B 5/02; A61B 5/026; A61B 5/0295

(52) U.S. Cl. CPC A61B 5/02225; A61B 5/0261; A61B 5/0295; A61B 5/02007; A61B 5/72
USPC 600/492

(57) ABSTRACT

Method to obtain continuous recording of the central arterial blood pressure waveform noninvasively utilizes dual (distal occlusion and proximal) brachial artery occlusion cuffs and dual external osculation. The distal arterial occlusion cuff eliminates venous stasis artifact and flow related gradient from aorta to the brachial artery. The proximal cuff measures, and delivers, dual external oscillation. The dual external oscillation allows measurement of the arterial compliance at a multitude of transmural pressure values during each cardiac cycle. Transmural pressure/arterial compliance and arterial pressure curves are subsequently reconstructed using dual external oscillation. The curves consist of two parts, rapid and slow parts, both at the frequency higher than the arterial pulse.

### CROSS REFERENCE TO RELATED APPLICATIONS

The invention described and claimed herein below is a Continuation-in-Part (CIP) application of U.S. patent application Ser. No. 12/234,168, filed on Sep. 19, 2008 (“Parent application”), and derives its basis for priority under 35 USC §119(a)-(d) from the Parent application, which is incorporated by reference herein.

### BACKGROUND OF THE INVENTION

Central blood pressure can be measured invasively in the ascending aorta. It determines myocardial afterload (impedance for blood outflow) and perfusion of the critical organs (brain, myocardium). Central blood pressure also determines both static and dynamic stress in the end organ vessels (carotids, coronaries, vertebral arteries), which eventually leads to degenerative changes of wear and tear. Due to the flow related pressure gradients, as well as pulse wave propagation and reflection in the complex arterial tree, peripherally measured pressure differs from the central one. In patients after cardiac bypass, systolic gradient measured invasively was 6.9+/−6.9 mm Hg and in 3 out of 8 patients exceeded 10 mm Hg (VanBeck, 1993). This difference is called central to peripheral pressure gradient.

Oscillometric blood pressure measurement in the brachial artery correlates with the central pressure in patients undergoing cardiac catheterization (older patients with cardiac disease) (Borow, 1982). It was shown to differ in younger patients (Wilkinson, 2001; Hulsen, 2006). Moreover there is no way to predict the cases, where brachial pressure differs from central pressure (Wilkinson, 2001). When brachial pressure is similar to central pressure, distal artery occlusion abates the flow but does not significantly change pulse pressure. However in patients with significant aorto-brachial pressure gradient after cardiac bypass, forearm cuff, inflated above systolic pressure, was shown to eliminate aorto-brachial pressure gradient measured invasively (Katsuno, 1996). Similarly wrist compression diminished radial to aorta pressure gradient (Pauca, 1994)

In 1931, Von Recklinghausen described a dual cuff (occlusive and sensing) technique, using aneroid valves in series. This ‘oscillotonometer’, was set in a sealed black box and provided a visual measure of systolic, diastolic and mean arterial pressures. Similar apparatuses are commercially available. Distal cuff is used as oscillatory sensor in these devices.

There is no universally accepted method to measure central blood pressure noninvasively. There is no noninvasive central blood pressure measurement method which would reliably reconstruct central blood pressure waveform when pulse is irregular, weak or absent (in the patients with left ventricular assist device or during cardiac arrest).

There is no noninvasive blood pressure measurement device which would work during cardiopulmonary resuscitation to monitor adequacy of chest wall compressions and detect return of spontaneous circulation.

Maintaining arterial relaxation pressure above 20 mmHg during cardiopulmonary resuscitation is recommended in Advanced Cardiac Life Support guidelines by American Heart Association to maintain coronary perfusion pressure and increase chances of successful resuscitation. However there is no noninvasive blood pressure device which could measure this pressure.

Commercially available carotid or radial artery applanation tonometer produced by Sphygmocor uses pulse wave analysis and pulse wave transfer function to estimate central blood pressure (Hirata, 2006). Epidemiological studies performed with this device demonstrated that central blood pressure elevation and widening of the pulse pressure correlates with an increased blood pressure, which in turn is associated with increased morbidity/mortality. The drawback of applanation tonometry comes from its inability to be performed on all patients (like in patients with weak or absent peripheral pulses). Moreover the method is operator dependant (requires acquisition of a high fidelity pulse tracing) and requires specialized training. Technique is semi quantitative and needs independent calibration. When cuff pressure is used to calibrate the pressure, central pressure assessment by pulse wave analysis was shown to be worse from cuff pressure measurement (Cloud, 2003).

Sharir, et al. (1993) validated noninvasive method to assess central blood pressure previously described by Marmor, et al. (1987). The method involved measuring the time delay between the R wave of the electrocardiogram (ECG) and the brachial pulse during gradual deflation of an arm cuff. The delay shortened with declining cuff pressure, enabling pressure-time data for the ascending limb of the arterial pressure wave to be estimated. Sharir used a computer controlled occlusive cuff, a brachial artery Doppler probe and ECG gating. Once central pressure equals or exceeds cuff pressure, flow can be registered in the brachial artery. By gradually increasing cuff pressure and registering ECG R wave gated interval up until the appearance of flow, authors reconstructed the upstroke of central blood pressure pulse.

The down side of such known method is that it requires that measurements be performed over multiple cardiac cycles, requiring special equipment and that the measurements cannot be obtained in patients with significant beat to beat central pressure variation and arrhythmias (such as atrial fibrillation). Moreover, only the ascending part of pulse wave can be estimated with this method.

For that matter, an ability to follow variability of the arterial blood pressure waveform over time allows one to follow an interaction between the cardiac output, vascular resistance and vascular compliance. It is preferable in the aforementioned techniques that arterial blood pressure waveform should be not a peripheral, but central. The central arterial blood pressure should be recorded as proximal to the heart as possible. Additionally, central arterial blood pressure should be precise, i.e., mirror the exact recording of the arterial pressure which would be obtained by the invasive arterial catheter. It is known that the main source of errors when using noninvasive methods is artificially created venous stasis.

Many methods are known for the measurement of the blood pressure, but all have shortcomings such as an inability to measure blood pressure continuously, an inability to reflect central pressure waveform accurately, inherent inaccuracies related to the venous stasis and/or are invasive (without limitation).

One of the oldest blood pressure measurement methods is auscultatory noninvasive blood pressure (NIBP) measurement. NIBP registers Korotkov sounds during brachial cuff deflation, where their appearance and disappearance correspond to the systolic and diastolic blood pressure. NIBP, however, does not allow continuous measurement of blood pressure waveform, requires experienced operator to perform the measurements, and does not reflect central blood pressure. The venous artifact does not affect auscultatory method; there is no flow/sound from the venous system. The venous artifact affects oscillometric and volume clamp methods.

Oscillometric NIBP measurement devices register cuff oscillations caused by the arterial pulse and find their maximum; oscillatory maximum occurs when cuff pressure equalizes with mean arterial pressure. This is the point when pulse pressure oscillation induces the highest volume change. Although this method does not require an operator, it still does not allow continuous measurement of blood pressure waveform, does not reflect central blood pressure, and does not account for the error created by the venous stasis when blood pressure cuff is inflated, and does not measure, but rather estimate systolic and diastolic blood pressure values.

A volume clamp method utilizes variable external pressure with the plethysmographic feedback loop. Fixed transmural pressure allows tracing of the arterial waveform. However this waveform is not of the central arterial pressure, but peripheral blood pressure, and as such is mostly inaccurate, and highly susceptible to the external noise. Volume clamp method can not be used on the proximal artery due to venous artifact—venous pressure increases to a level of the cuff pressure and increases blood volume under the cuff.

Applanation tonometry registers transmural pressure through the flattened arterial wall. However the obtained waveform is not of a central blood pressure but peripheral blood pressure. Moreover, the obtained waveform is mostly inaccurate, highly susceptible to the external noise, and in some patients simply not obtainable. In attempt to reconstruct central blood pressure waveform, an arterial waveform obtained by applanation tonometry is transformed by the population based transfer function; however as any population based construct, such transfer function can not account for the individual outliers.

Most recent addition to the continuous pressure waveform recording methods, external oscillatory method by Penaz [Penaz J, Honzikova N, Jurak P. Vibration plethysmography: a method for studying the visco-elastic properties of finger arteries. Med Biol Eng Comput. 1997 November; 35(6):633-7, reconstructs arterial compliance curve using transmural pressure/volume relationship. In order to avoid venous pressure artifacts introduced by venous stasis, Penaz's volume clamp and external oscillometric method use a finger and not brachial cuffs. That makes the measurement even more distal from the central aorta and introduces additional artifacts not only due to pressure gradient from the aorta to the measurement site, but also due to the pressure wave reflections.

### SUMMARY OF THE INVENTION

The present invention overcomes the shortcomings of the known arts, such as those mentioned above.

The present invention provides systems and methods for automatically generating an accurate central blood-pressure measurement.

Using the present invention, not only are systolic and diastolic central blood pressure values measured, but the whole waveform is reconstructed.

The central blood-pressure measurement and waveform reconstruction is desirable to estimate cardiac work/contractility indexes, to measure stroke volume, to assess central circulation, stratify blood pressure related cardiovascular risk, etc. If blood pressure treatment is initiated, assessment of central blood pressure response to treatment is important.

In one embodiment, the invention provides a method to measure central blood pressure with the following characteristics:

(1) eliminates flow related blood pressure drop and pulse wave reflection from the distal vasculature—two main sources of discrepancy in pressures measured in brachial artery and aorta. Totally occluding eliminates an effect of distal venous stasis on blood volume under the cuff (venous artifact)

(2) is noninvasive, simple and easily performed by general practitioner without specialized training;

(3) is operator independent and applicable to a variety of patients regardless of their age or status of their hemodynamics;

(4) is based on the cuff blood pressure measurement, which is accepted standard and well known to the practitioners; and

(5) is based on simple physical principles and does not require validation studies in every population to check empirical assumptions, which may not be applicable to different populations;

(6) is particularly advantageous when pulse is irregular, weak or absent (for example, in the patients with left ventricular assist device);

(7) can be used during cardiopulmonary resuscitation to monitor adequacy of chest wall compressions and detect return of spontaneous circulation. Relaxation pressure of 20 mmHg or more is required to maintain coronary perfusion pressure and increase chances of successful resuscitation.

Outflow occlusion distal to the brachial artery eliminates flow related pressure drop, kinetic energy related pressure component and pulse wave reflection from the distal vasculature—three main sources of discrepancy in pressures measured in brachial artery and aorta. Outflow occlusion also releases endothelium derived vasodilatation factor, which operates to decrease flow related pressure gradient in the brachial artery.

Distal brachial artery occlusion also minimizes pressure gradient from the aorta to brachial artery due to temporary flow cessation and pulse wave reflection from the distal vasculature. This was demonstrated by Katsuno, 1996 using invasive measurements and distal brachial artery occlusion in cardiac bypass patients.

Brachial cuff inflated to a level below systolic blood pressure allows arterial inflow but blocks venous outflow. Increased venous pressure approaches cuff pressure and interferes with measurements using brachial blood volume. (FIG. 15). Distal cuff inflation above systolic pressure eliminates venous artifact.

Heretobefore, there was no noninvasive method, and system for implementing the method designed to measure brachial artery segment proximal to a distal occlusion. Auscultatory or palpatory methods cannot be used as there is no flow through the artery. Oscillometric method is not validated for measuring pressure in the brachial artery with distal occlusion.

The inventive system and method operate to improve a central aortic blood pressure approximation with brachial artery pressure using distal occlusion and measure proximal pre-occlusion brachial artery pressure noninvasively using extrinsic perturbation, as described in detail below.

The invention allows or provides for many desirable characteristics of the blood pressure monitoring method, i.e., performs continuous recording of the arterial waveform, insures that the recording reflects central arterial waveform, eliminates venous stasis artifact and allows for monitoring noninvasively and automatically, without any need for a trained operator.

### BRIEF DESCRIPTION OF THE DRAWINGS

Further features and advantages of the invention will become apparent from the description of embodiments that follows, with reference to the attached figures, wherein:

FIG. 1 shows noninvasive blood pressure measurement device 10 connected to a pressure measurement cuff 20 and distal artery occlusion cuff 150;

FIGS. 2 A and 2B together show how a reflected pressure wave is eliminated with arterial occlusion;

FIG. 3 illustrates using windkessel circulation model how occluding distal brachial artery (represented by open switch) leads to the equilibration of systemic and brachial pressures (voltage in the windkessel model);

FIG. 4 shows blood pressure measurement algorithm using extrinsic oscillation, and distal artery occlusion where blood pressure equals to external compression pressure Pe with maximal compliance Cmax;

FIG. 5 shows blood pressure measurement algorithm when the plurality of compliance maximums is obtained during the measurement of pulsatile or variable blood pressure and minimum, maximum and mean values of central blood pressure are displayed;

FIG. 6A shows that pressure in the arterial segment proximal to occlusion is measured using extrinsic perturbation. Maximal induced arterial oscillation is registered when Pe=Pa;

FIG. 6B shows that maximal calculated compliance is found when Pa=Pe;

FIG. 7A shows superimposed induced (extrinsic) and arterial pulse related (intrinsic) oscillations;

FIG. 7B shows the plurality of compliance maximums when arterial pressure fluctuates between maximal (systolic) and minimal (diastolic) values;

FIG. 7C shows a standard way of measuring oscillometric blood pressure; i.e., small oscillations; there is no extrinsic oscillation, visible oscillations coming from arterial pulse.

FIG. 8A depicts plots of Arterial pressure Pa, arterial volume Va and cuff pressure Pe with overlapped high and low frequency cuff Pe oscillation;

FIG. 8B depicts fluctuations of arterial pressure, cuff pressure, and arterial volume after application of dual frequency extrinsic oscillation;

FIG. 9 depicts a comparison of actual (“x's”) and estimated (solid line) blood pressure increments ΔPa;

FIG. 10 depicts a comparison of actual (+ sign) and estimated (solid) increments of the transmural pressure ΔPtm over time interval Δt.

FIG. 11 depicts a comparison of the actual transmural pressure Ptm and interval sum of estimated transmural pressure increment Sum(ΔPtmᵢ*Δtᵢ);

FIG. 12 depicts Comparison of actual (dots) and estimated (solid line) compliance. Estimate approximates actual values;

FIG. 13A depicts compliance/transmural pressure relationship. Estimated compliance is shifted on the X axis;

FIG. 13B depicts compliance and transmural pressure relationship after subtracting −32.6 to align the maximum compliance with zero Ptm;

FIG. 14 depicts Estimated and actual arterial pressure waveforms;

FIG. 15 depicts venous artifact which could arise during cuff inflation, i.e., where venous pressure (CVP) increases close to diastolic pressure during cuff inflation; and

FIG. 16 depicts one embodiment of a vibrator, accelerometer and pressure sensor under the cuff and above the artery, for use with the inventive system and method.

### DETAILED DESCRIPTION OF THE INVENTION

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

In an embodiment, the invention provides a method to obtain continuous recording of the central arterial blood pressure waveform noninvasively utilizes dual (distal occlusion and proximal) brachial artery occlusion cuffs and dual external oscillation. The distal arterial occlusion cuff eliminates venous stasis artifact and flow related gradient from aorta to the brachial artery. The proximal cuff measures, and delivers, dual external oscillation. The dual external oscillation allows measurement of the arterial compliance at a multitude of transmural pressure values during each cardiac cycle. Transmural pressure/arterial compliance and arterial pressure curves are subsequently reconstructed using dual external oscillation. The curves consist of two parts, rapid and slow parts, both at the frequency higher than the arterial pulse.

Alternatively, the oscillator/pressure sensor and accelerometer, under the proximal edge of the pulse, induce and measure rapid oscillation. Concurrently, overlying cuff compresses veins, distal to the measurement site, and induces slow oscillation for the purpose of obtaining multitude of transmural pressure readings during each cardiac cycle. Continuous central blood pressure measurement device works even when arterial pulse is weak, irregular or absent. It can be equally successfully used to monitor effectiveness of chest compressions and return of spontaneous circulation during cardiopulmonary resuscitation, just as to monitor arterial blood pressure waveform in the ambulatory setting, as an attachment to smart phone.

### REFERENCE CHARACTERS

Pe: External (measuring cuff) pressure (mmHg)
Pocclusion: pressure in the cuff occluding distal artery (mmHg)
Pa: Arterial pressure (mmHg)
V: Blood volume under the proximal measuring cuff
Posc: Extrinsic pressure Pe oscillation
Vosc: Induced blood volume V oscillation
C: Compliance, C=−Vosc/Posc
Cmax: Maximal compliance (when Pe=Pa)
Ca: arterial compliance (ml/mmHg): Ca=dVa/dPtm; Ca=max, when Ptm=0; Ptm: transmural pressure (mmHg), Ptm=Pa−Pe;
Psyst: systolic arterial pressure (mmHg)
Pdiast: diastolic arterial pressure (mmHg)
dVc_e_slow, dPe_slow:
   cuff volume and pressure change caused by slow external oscillation (40 Hz>slow_frequency>1 Hz)
dVc_e_fast, dPe_fast:
   cuff volume and pressure change caused by fast external oscillation
10: Blood pressure measurement apparatus
15: Distal occlusion cuff
20: Inflatable proximal (measurement) pressure cuff
30: Proximal brachial artery with blood volume V
40: Oscillator for repetitive cuff pressure perturbation Posc
50: Manometer (pressure sensor) for sensing cuff pressure (Pe)
60: Blood volume V sensor (plethysmograph)
70: CPU for data acquisition, occlusion and measurement cuff
control, data processing, compliance C calculation display and user control execution
80: Display
90: Body portion containing proximal blood vessel
100: Cuff connecting hoses
110: Pump and valves for cuff pressure control
120 User controls
130: Aortic arch
140: Brachial artery occlusion

In an embodiment, noninvasive blood pressure measurement apparatus 10 consists of the distal occlusion cuff 15, means 20 (proximal measuring cuff (or proximal inflatable pressure cuff) to variably compress the vessel 30. The distal occlusion cuff is inflated above systolic blood pressure and then the proximal cuff is used to measure the brachial pressure using an oscillatory method with extrinsic perturbation, as described herein.

Extrinsic oscillator 40 introduces cyclical pressure perturbation (Posc) to the proximal vascular bed 30. Pressure sensor 50 senses extrinsic vascular bed compression force (Pe) and occlusion pressure (Pocclusion). Volume sensor 60 senses vascular bed volume response to extrinsic cyclical perturbations. Processing unit 70 and display unit 80 also are included.

As shown, proximal inflatable pressure cuff 20 is placed around the patient's extremity 90 and is connected via one or more connecting hoses 100 to a measuring apparatus 10. Pressure cuff 20 is connected to the pump 110, oscillator 40, pressure sensor 50 and volume sensor 60. Distally to the measurement cuff 20, occlusion cuff 15 is placed around the extremity 90 and connected via connecting hose 100 to the measuring apparatus 10. Occlusion cuff 15 is connected to the pump 110 and pressure sensor 50 and maintains a pressure sufficient to occlude the vascular vessels distal to its position on the patient. Processing unit 70 is connected to pressure sensors 50, volume sensor 60, pressure pumps 110, oscillator 40, display 80 and user controls 120.

Preferably, occlusion cuff 15, pump 110 and cpu 70 are configured to cooperate in order to maintain occlusion cuff 15 functionally as an occluding device only, that is, occlusion cuff 15 only operates to occlude.

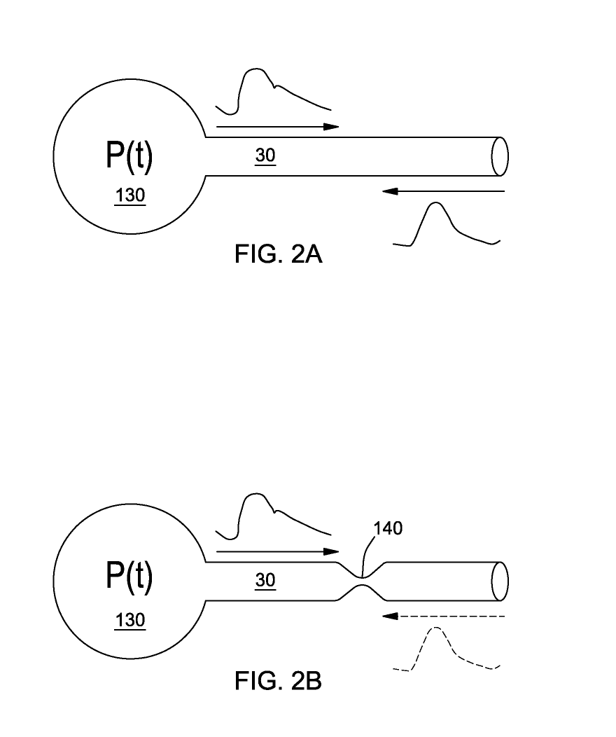

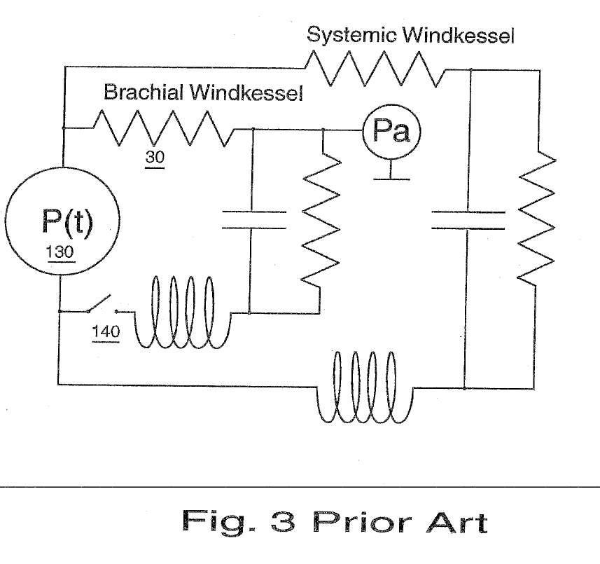

Operation—FIGS. 2, 3

FIG. 2A illustrates how pressure in the aortic arch 130 is distorted by the reflected pressure wave returning from the arterial branches distal to the measurement site. Occlusion of the artery 140 distal to the measurement site in FIG. 2B eliminates locally reflected pressure wave (see “ghost” wave).

In FIG. 3, central and peripheral (brachial) circulation is represented by two parallel electric windkessel equivalents. Measuring pressure (voltage) in the brachial circuit Pa is not equivalent to the pressure measurement in the central circuit P(t). This is due to the pressure drop across resistive, inductive and capacitance components in the brachial circuit. Accounting for that using “ideal” transfer function allows central blood pressure estimation but does not account for impedance variation in different patients. Occluding brachial artery distally (opening switch 140 in the brachial circuit) eliminates pressure drop across resistive, inductive and capacitance components of the brachial circuit and allows to measure central blood pressure: Pa=P(t).

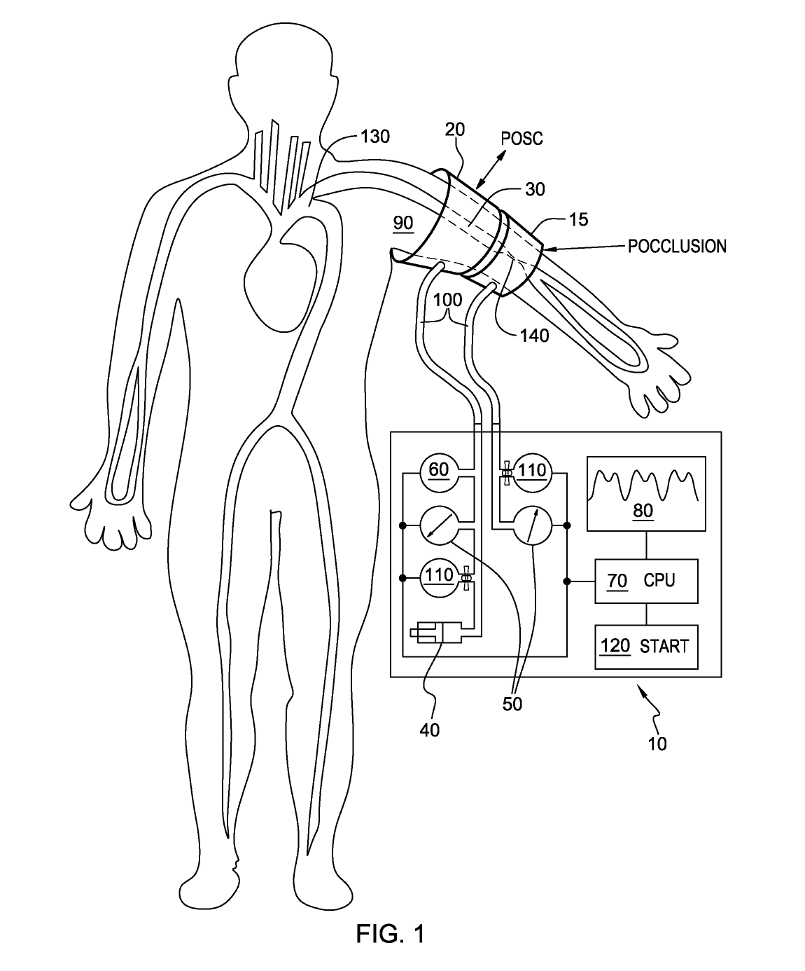

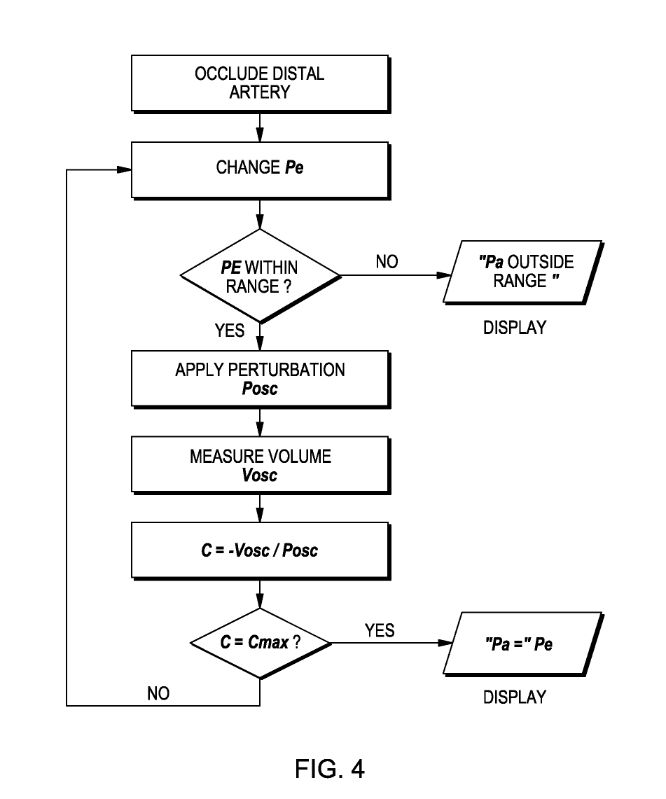

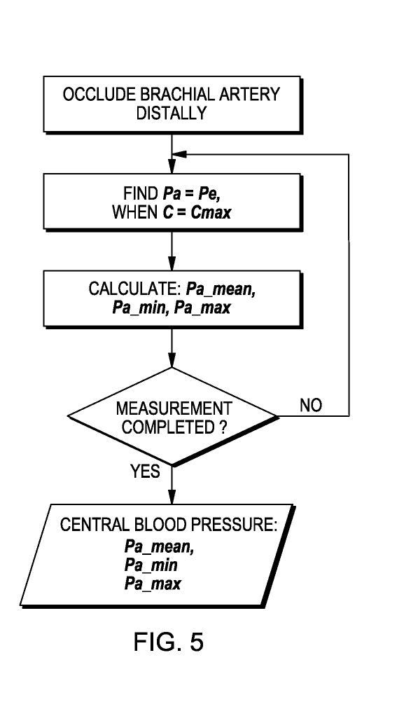

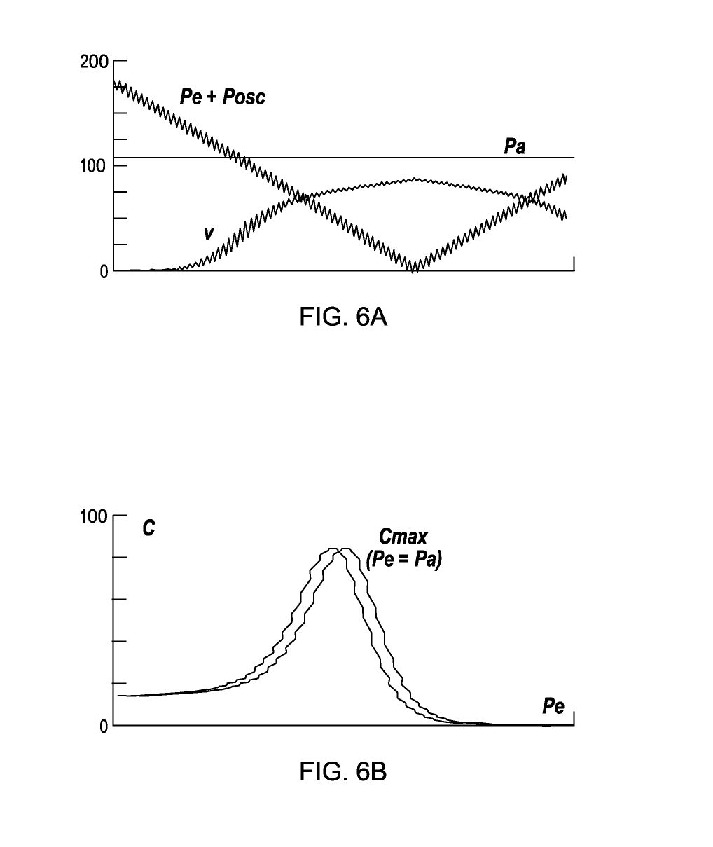

Operation—FIGS. 1, 4-7

In an embodiment, to measure the blood pressure Pa, pneumatic occlusion cuff 15 and proximal pressure measurement cuff 20 are fitted around the extremity 90 and attached via the connecting hoses 100 to the measuring unit 10. Occlusion cuff 15 is inflated above estimated systolic pressure to Pocclusion and maintains the pressure at Pocclusion during testing. Pressure cuff 20 is gradually inflated with the pressure pump 110 (Pe). While pressure Pe in the cuff 20 is varied by the pressure pump 110, oscillator 40 adds an extrinsic oscillatory component Posc. Pressure Pe is measured in the cuff 20 by the pressure sensor 50. Pressure sensor 50 reads average pressure (e.g. using low pass filter) and oscillatory pressure component Posc (e.g. high pass filter). Blood volume under the cuff V is measured with volume sensor 60. Oscillatory volume component is measured as Vosc using high pass filter or pressure and volume signal cross correlation. In another embodiment oscillator 40 is a sound wave generator and pressure sensor 50 is a microphone.

As the proximal measurement cuff 20 is inflated with the pump 110, Posc is applied and vessel compliance C is calculated as C=Vosc/Posc. For that matter, the proximal measurement cuff 20 is inflated to cover the expected arterial pressure range.

In more detail, while cuff pressure Pe is being changed, oscillatory pressure and volume components are measured and compliance C=−Vosc/Posc is calculated.

Vascular compliance C is maximal (C=Cmax) when the cuff pressure Pe approximates mean vascular pressure and transmural pressure=0. When vascular bed is collapsed (Pe>>Pa), C becomes zero.

To assess vascular compliance C, high fidelity measurements are taken over the range of Pe. C=Cmax when Pe=Pa.

When arterial pressure is pulsatile or varies over time, plurality of compliance peaks C=Cmax at different external pressure Pe values are obtained. Cmax at highest external pressure Pe corresponds to high (systolic) and at lowest Pe corresponds to low (diastolic) arterial blood pressure.

Multiple alternative inventions embodiments are possible depending on the vascular bed compression method 20, extrinsic perturbation mode 40 (vibration, acoustic wave, etc.), receiving volume sensor 60 modality and placement.

In an alternative embodiment, cuff 20 may be filled with liquid (to diminish cuff compliance) and used to compress the proximal brachial artery 30.

In another embodiment, compression is performed applying direct pressure over the proximal artery with a tonometer. Using tonometry pressure is applied to the tissue covering the vessel or compartment rather than around the extremity.

In alternative embodiments, oscillator 40 utilizes electromechanical pneumatic, piezo, vibratory or acoustic perturbation.

In alternative embodiments, oscillator 40 is located directly over the body part containing the vessel, combined with a vessel compression device 20 or over the body part distant from compression device 20.

In alternative embodiments, volume sensor 60 senses changes in pressure in the cuff, volume in the cuff, Doppler signal (from blood or blood vessel wall), optical signal (e.g. scattering or border recognition), plethysmogram (photo, impedance, etc).

In alternative embodiments, volume sensor 60 and pressure sensor 50 are close to the cuff or incorporated in the cuff 20. Closer placement of the oscillator/sensor diminishes lag for cuff compliance measurement and vascular compliance estimation.

In an alternative embodiment, extrinsic perturbation measuring unit is incorporated into standard NIBP measurement machine.

Commonly used NIBP machines are based on the oscillatory measurement method and changes Pe, while registering intrinsic oscillations. When Pe=Pa, oscillation amplitude reaches maximum. Attaching additional extrinsic oscillation measuring unit 10 to the NIBP hose/cuff connection allows incorporating extrinsic oscillations to assess vascular pressure. Pe is varied by the noninvasive machine; Posc is introduced, volume response Vosc is registered and compliance C=−Vosc/Posc is calculated. Compliance/pressure dependence is obtained C (Pe) in the measured range of Pe. Preferably, external oscillations do not interfere with intrinsic oscillation registration (e.g. they are different frequency range). Distal artery occlusion using this approach allows measurement of central blood pressure.

The inventive systems and methods for noninvasive central pressure measurement are advantageous for at least the following reasons.

Central blood pressure can be measured in the absence of pulsatile flow with distal cuff occlusion.

Central Blood Pressure can be measured when blood pressure pulsation is very weak (shock, premature neonates).

Blood pressure can be measured when blood pressure pulsation is irregular (arrhythmias) or changes rapidly.

Blood pressure can be measured faster as it does not require extending the measurement over few cardiac cycles.

Blood pressure can be measured at both low and high pressure values.

Blood pressure can be measured in critically ill or trauma patients with hemodynamic instability. Blood pressure can be measured during cardiopulmonary resuscitation to ensure that chest compressions are adequate and maintain arterial relaxation pressure above 20 mmHg.

Blood pressure can be measured during cardiopulmonary resuscitation to detect return of spontaneous circulation and to measure blood pressure during arrhythmias.

The inventive method is automatic and does not require specialized training from the operator.

The inventive method avoids invasive arterial pressure monitoring for many patients and provides backup monitoring capability for others.

The inventive method is based on simple physical principles and does not require assumptions about ideal transfer function.

Through the use of extrinsic perturbation and distal artery occlusion, the inventive systems and methods eliminate the pressure gradient between brachial and central blood pressure and allow for measuring the central blood pressure noninvasively. The inventive method is devoid of limitations of current noninvasive central pressure measurement methods. The inventive method does not make assumptions about central to peripheral transfer function. With distal artery occlusion it simply eliminates brachial-central pressure gradient.

Pressure measurement using extrinsic perturbation does not require presence of the pulsatile flow and facilitates measuring the pressure in the brachial artery proximal to the occlusion which corresponds to central blood pressure.

It is simple to apply, does not require specially trained personnel. The systems and methods can be used during transport/evacuation, in the hospital, ambulatory setting or patient's home.

In one form, the inventive device or system comprises (a) pressure application means 20 for applying an external pressure to a portion of the pressing body portion containing a blood vessel to assert an external pressure in the blood vessel, vessel compression means 15 for applying pressure to the body portion and occluding the blood vessel arranged distally to the pressure application means. Pressure changing means in the pressure application means change pressure level across a range which is expected to include blood pressure level, repetitive pressure perturbation means superimpose a pressure perturbation onto the external pressure already established in the blood vessel by pressure level in the pressure application means and pressure sensing means for sensing the external pressure applied by in the pressure application means.

Vessel volume measurement means measure blood vessel volume at the body portion location of under the pressure application means, compliance calculating means calculate compliance as a ratio of the blood vessel volume change to the pressure perturbation at the each external pressure level over a varying pressure range applied by in the pressure application means and means for indicating that the external pressure level at the maximal compliance calculated is the central blood pressure as the cuff pressure level, where the compliance is maximal.

Preferably, the pressure application means 20 comprises an inflatable pressure cuff. For that matter, the inflatable pressure cuff is configured for pressing body portion and can be filled with a noncompliant fluid. The means of repetitive pressure perturbation is electromechanical, the vessel volume measurement means comprises a pressure sensor under said pressure application means are pressure measurement means in the inflatable pressure cuff and the pressure application means applies a varying pressure to the body portion. The applied varying pressure is in a range beginning at a pressure level that is less that systolic blood pressure and ending in a range that exceeds systolic blood pressure. In addition, the pressure application means eliminates any pressure gradient that might normally exist between the body aortic arch and body portion location of the pressure application means. In many cases, the blood vessel is the brachial artery and the blood pressure is measured in the segment of the brachial artery proximal to the occlusion.

### Distal Occlusion Cuff:

To eliminate pressure gradient from aorta to the measurement site, distal occlusion cuff 15 is inflated above systolic pressure. Apart from eliminating arterial pressure gradient, implementing such occlusion avoids venous congestion which changes pressure/volume relationship of the arm and introduces artifact to oscillometric, volume clamp and external oscillometric methods. Thus, central blood pressure now can be measured with all pressure/volume measurement methods.

### External Oscillation:

Adding external oscillation makes oscillometric methods less dependent on the beat to beat arterial waveform fluctuations, but does not allow reconstruction of the arterial waveform. To overcome this limitation, external oscillation can be applied and the arterial waveform reconstructed, as described below.

Cuff Pressure Change Speed:

Cuff pressure is commonly increased rapidly, for example, to between 140 and 220 mmHg, and then slowly released 1-2 mm Hg/s to register pulses over variety of transmural pressures. Thus, registering blood pressure takes time and pulse waveform is not reconstructed.

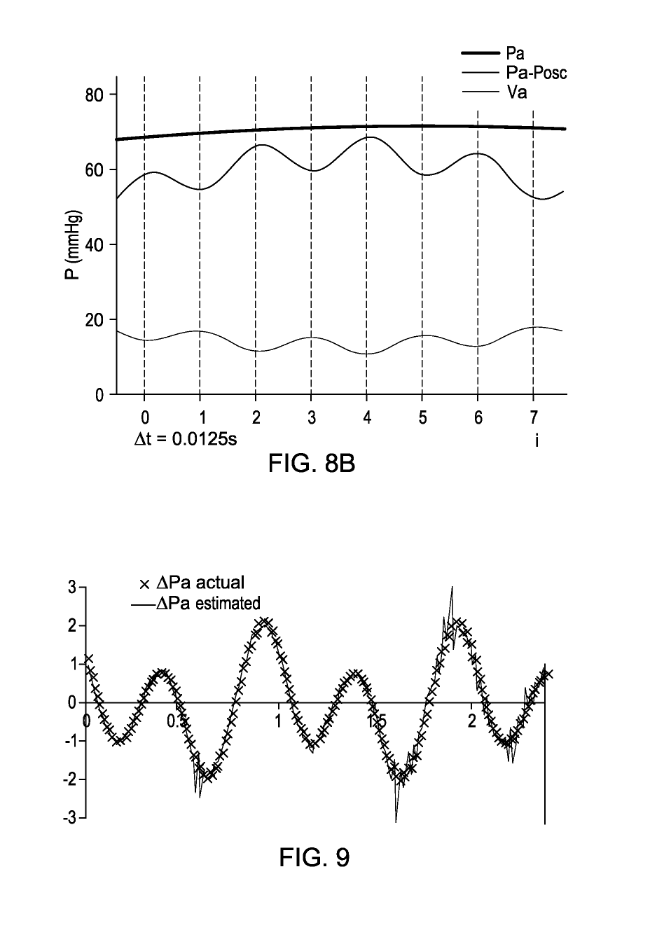

Fluctuating the cuff pressure at a higher frequency than Pa(t), as shown in FIGS. 8A and 8B, changes allows for registration of multiple points of transmural pressure over short period of time. Adding a second oscillation at a higher frequency enables that multitude compliance measurements can be taken while transmural pressure changes over series of values created by the first oscillatory wave.

Superimposing low and high frequency oscillations on the cuff pressure allows that arterial compliance can be calculated for each high frequency oscillation pulse: ΔVa/ΔPe_fast and for each change in baseline between two successive oscillations: Δ(ΔVa/ΔPe_fast)/ΔPe. Thus, the relationships ΔVa/ΔPe(Pe) and Ca(Pe) are obtained and used to reconstruct Ca(Ptm); reverse function Ptm(Ca) and Ca(t). Then, the arterial waveform is reconstructed as:

Pa(t)=Pe(t)−_{Ptm}(Ca(t))

This calculation is carried out with time interval Δt resolution equal to one external oscillation half period.

FIG. 8A depicts plots of Arterial pressure Pa, arterial volume Va and cuff pressure Pe with overlapped high and low frequency cuff Pe oscillation. Cuff oscillation Pe is transmitted to the artery and registered as Va oscillation.

FIG. 8B depicts fluctuations of arterial pressure, cuff pressure (extrinsic), and arterial volume after application of dual frequency extrinsic oscillation. That is, preferably, the cuff or extrinsic pressure is changed slowly over time to realize multiple transmural pressures. Concurrently, a low frequency (or slow) oscillatory signal is generated at a frequency that is greater than the heart rate, that rides on the slowly changing Pe. Preferably, a second (or fast) oscillatory signal is generated at a frequency that is twice the frequency of the first or slow oscillatory signal.

Measurements of relative arterial volume Vaᵢ and cuff pressure Peᵢ i=0 . . . 7 are obtained at each time moment tᵢ which corresponds to the extrema (minimum and maximum) of high frequency oscillation starting at i=0. Half-period of oscillation is the time interval Δt between two successive measurements: Δt=tᵢ−tᵢ₋₁=0.0125 s (FIG. 8B). Increasing or decreasing oscillation frequency Δt decreases or increases. Δt can be selected from a wide range (infrasonic, sonic, ultrasonic).

Derivation of how arterial pressure increment between i−1 and i is obtained in equation (4), below.

Cuff pressure increment ΔPe at time tᵢ:

ΔPeᵢ=Peᵢ−Peᵢ₋₁

Arterial pressure increment ΔPa at time tᵢ:

ΔPaᵢ=Paᵢ−Paᵢ₋₁.

Arterial volume increment ΔVa at time tᵢ:

ΔVaᵢ=Vaᵢ−Vaᵢ₋₁.
Arterial compliance:

Caᵢ=ΔVaᵢ/(ΔPaᵢ−ΔPe+ᵢ), (1)

Caᵢ₊₁=ΔVaᵢ₊₁/(ΔPaᵢ₊₁ΔPe₁₊₁). (2)
If time interval between i and i+1 moments Δt=tᵢ−tᵢ₋₁ is short, CaᵢøCaᵢ₊₁ and ΔPaᵢ₊₁≈ΔPaᵢ. Then,

ΔVaᵢ/(ΔPaᵢ−ΔPeᵢ)=ΔVaᵢ₊₁/(ΔPaᵢ−ΔPeᵢ₊₁),

(ΔPaᵢ−ΔPeᵢ)*ΔVaᵢ₊₁=(ΔPaᵢ−ΔPeᵢ₊₁)*ΔVaᵢ. (3)

From (3):

ΔPaᵢ=(ΔPeᵢ*ΔVaᵢ₊₁−ΔPeᵢ₊₁*ΔVaᵢ)/(ΔVaᵢ₊₁−ΔVaᵢ). (4)

Formula (4) approximates pressure fluctuations well, as is shown in FIG. 9, which compares actual (“x's”) and estimated (solid line) arterial pressure increments ΔPa over time interval Δt.

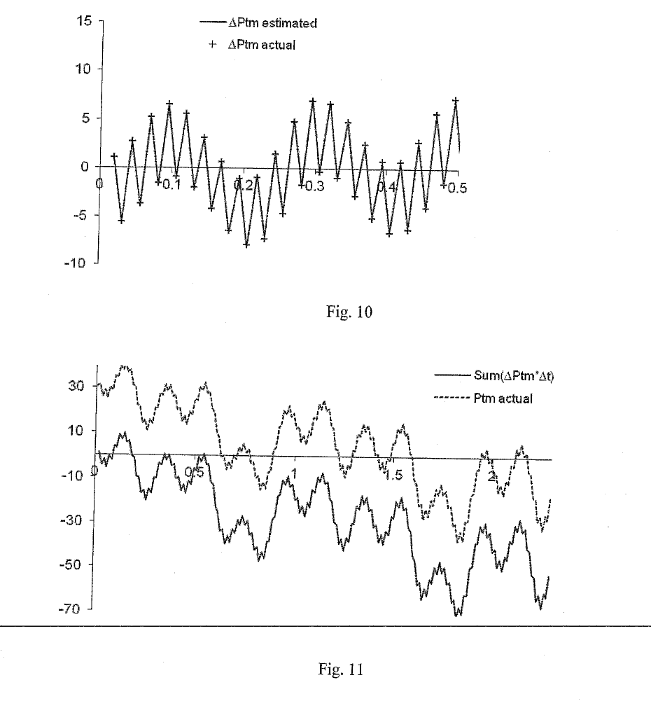

Once the increment of arterial pressure ΔPa is known, the increment of transmural pressure ΔPtm (FIG. 10) is obtained:

ΔPtmi=ΔPai−ΔPei (5)

FIG. 10 depicts comparison of actual (solid line) and estimated (“+'s”) transmural blood pressure increments ΔP.

Once we know the increment of transmural pressure ΔPtm over time interval Δt, we can obtain a sum of this increment Sum(ΔPtm*Δt) (FIG. 11).

FIG. 11 depicts a comparison of the actual transmural pressure Ptm and interval sum of estimated transmural pressure increment: Sum(ΔPtmᵢ*Δtᵢ). Because the initial value of Ptm₀ is unknown before the summation, curves are shifted, but form of the estimated Ptm closely follows actual Ptm.

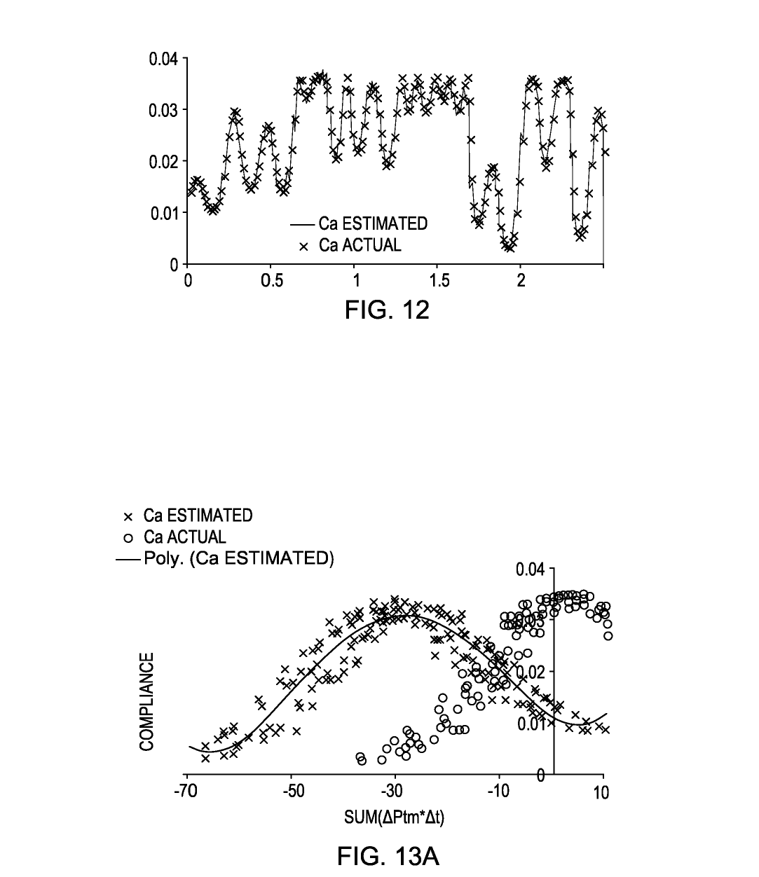

Once the increment of the transmural pressure ΔPtm is determined, compliance is estimated using formula 6 and plotted over time (FIG. 12).

Cai≈ΔVai/ΔPtmi. (6)

That is, FIG. 12 depicts a comparison of actual (dots) and estimated (solid line) compliance. Estimates approximate actual values.

Now, using the Ptm integral estimate (FIG. 11) and Ca estimate (FIG. 12), the relationship between compliance and transmural pressure Ca(Ptm) can be plotted:

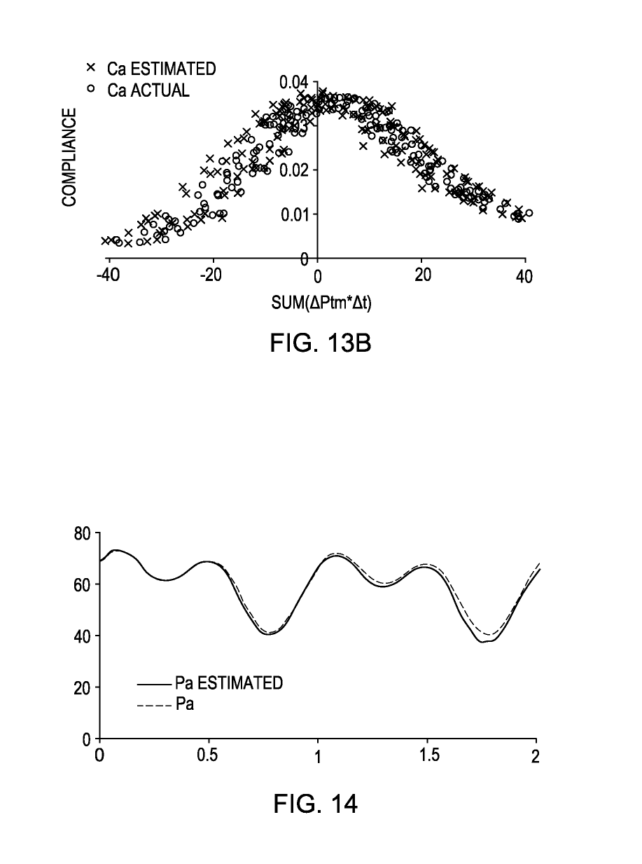

FIG. 13A depicts compliance/transmural pressure relationship. Estimated compliance is shifted on the X axis.

Then, the transmural pressure/compliance relationship is approximated by the polynome Poly., FIG. 13A uses a least square method and shifted along the X axis to align maximum compliance with zero transmural pressure. The value of this shift becomes a calibration factor, which has to be subtracted from each point, in order to align maximal compliance with the zero transmural pressure.

FIG. 13B depicts a relationship between compliance and transmural pressure after subtracting −32.6 to align the maximum compliance with zero Ptm.

Once the transmural pressure/compliance curve is estimated, it is used to assess Paᵢ:

Paᵢ=Peᵢ−Ptmᵢ.

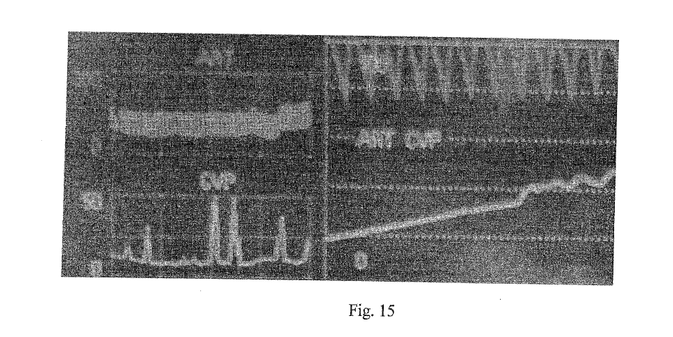

FIG. 14 depicts estimated and actual arterial pressure waveforms, where FIG. 15 depicts venous artifacts during cuff inflation. Venous pressure labeled as CVP increases close to diastolic pressure during cuff inflation; see trend over 1 hour on the left and 15 second recording on the right.

In an embodiment, the inventive method includes:

Applying the inflatable cuff 20 to the brachial artery, e.g., as a means to compress brachial artery.

Applying a distal occlusion cuff 15 to eliminate venous artifact.

Detecting arterial volume change, for example, by use of an accelerometer above the artery, a plethysmogram (photo, strain gauge, air, and impedance), ultrasound/Doppler, etc. inflating or deflating the inflatable cuff over a range of pressures that includes mean arterial pressure.

Preferably, applying a low frequency oscillation at the frequency exceeding heart rate to the cuff 20, so that a range of transmural pressures can be obtained repeatedly during each cardiac cycle.

Applying oscillation at the higher frequency at least double the low frequency oscillation (if used) to the artery with a period T. In the example 40 Hz oscillation was used.

Obtaining cuff pressure and relative arterial volume values twice per period at time points i, i=0, 1, . . . where tᵢ corresponds to the maximum or minimum of the high frequency Oscillation and time interval between two measurements Δt=tᵢ−tᵢ₋₁.

Estimating ΔPai per (4) for each time point tᵢ;

ΔPaᵢ=(ΔPeᵢ*ΔVaᵢ₊₁−ΔPeᵢ₊₁*ΔVaᵢ)/(ΔVaᵢ₊₁−ΔVaᵢ) (4)

Estimating ΔPtmᵢ per (5) for each time point tᵢ;

ΔPtmᵢ=ΔPaᵢ−ΔPeᵢ (5)

Estimating per (6) for each time point tᵢ.

Caᵢ=ΔVaᵢ/ΔPtmᵢ (6)

Plotting Caᵢ against sum of ΔPtmᵢ*Δtᵢ and shifted on X axis to align with the reference compliance curve so that maximum compliance corresponds to zero transmural pressure. This gives transmural pressure Ptm, for each time point ti.

Calculating arterial pressure as Paᵢ=Peᵢ−Ptmᵢ for each time point tᵢ. The arterial blood pressure waveform is reconstructed by the invention based thereon.

In addition to the hardware/software environment described above, a different aspect of the invention includes a computer-implemented method for performing the above method. As an example, this method may be implemented in the particular environment discussed above. Such a method may be implemented, for example, by operating a computer, as embodied by a digital data processing apparatus, to execute a sequence of machine-readable instructions. These instructions may reside in various types of signal-bearing storage media.

For example, the invention may be implemented by an apparatus that operate together to continuously measure a patient's central blood pressure according to the inventive principles. The method includes attaching a cuff to a measurement site on the patient; occluding an artery and/or the artery's branches distal to the measurement site; registering a blood volume in tissue at the measurement site; applying a variable external pressure to the cuff at the measurement site in order to maintain a constant blood volume in tissue at the measurement site; and estimating blood pressure in the measurement site to be equal to the applied variable cuff pressure.

Advantages

Distal occlusion cuff 15, which operates to occlude distal to inflatable cuff 20 by cooperation of pump and processor controlling same. Such occlusion only eliminates blood pressure gradient from the aorta, but also excludes distal veins and eliminates increased venous pressure contribution to the blood volume under the cuff. Such operation makes feasible noninvasive diastolic pressure measurement during CPR as well as volume clamp and external oscillatory methods.

The arterial compliance/transmural pressure curve is bell shaped (see FIG. 13A), thus reverse solution (transmural pressure from compliance) is non-unique. For example, the same single point of compliance can be observed at negative or positive transmural pressure. Consequently, a segment of compliance curve with different transmural pressures has to be analyzed. Dual oscillation technique allows the measurement of Ca(t), dCa(t)/dPe(t) and reconstruct Pa(t) with time resolution of external oscillation period.

Combined vibrator/accelerometer/pressure sensor allows for the measurement of external oscillatory blood pressure using standard cuff and can reconstruct Pa(t) using oscillation in the standard cuff.

Arterial pressure volume relationship Va(Ptm), where Ptm=Pa−Pe, is known to have sigmoid shape. Derivative of this relationship is arterial compliance Ca(Ptm).

Ca(Ptm) is bell-shaped with maximal compliance occurring at the zero transmural pressure.

Standard oscillometric NIBP measurement method uses intrinsic cuff pressure oscillation caused by pulse pressure.

If cuff volume is known, one can calculate dVa_pulse=dVc from dPe_pulse using plethysmographic formula:

dVc=dVa_pulse=Vc*dPe/Pe

Vc is not generally known, but maximal dPe coincides to maximal arterial compliance Ca when Ptm=0.

Using external cuff oscillation external oscillation is introduced to the cuff and arterial volume change is registered.

Arterial compliance is measured using external perturbation. Maximal induced arterial volume oscillation happens at the point when Pe=Pa. Pe (external) is used interchangeably herein with Pc (cuff), i.e., Pc=Pe. Put another way, external pressure Pe is applied with the cuff, which has a cuff pressure Pc.

External oscillation principle was successfully tested by Penaz J, Honzikova N, Jurak P. Vibration plethysmography: a method for studying the visco-elastic properties of finger arteries. Med Biol Eng Comput. 1997 November; 35(6):633-7, on the finger.

In clinical practice, blood pressure is most commonly recorded at the brachial artery. The Penaz method of external oscillation does not work at the brachial artery as the occlusion cuff occludes not only arteries, but veins also. While cuff pressure is below systolic, limb has inflow, but no outflow, until venous pressure reaches cuff pressure. Thus external oscillation may detect systolic pressure, but fails to detect diastolic pressure as venous pressure is close to diastolic.

Diastolic pressure determines coronary inflow pressure. In CPR, diastolic pressure above 20 mmHg is an indicator of effective compressions and is required to improve chances of successful resuscitation. Heretobefore, there is no non-invasive field-applicable device to measure diastolic blood pressure or detect intrinsic pulse with diastolic pressure above 20 mmHg.

As already mentioned, the distal occlusion cuff eliminates venous contribution to the blood volume changes and makes feasible noninvasive diastolic blood pressure measurement even in the absence of effective cardiac output.

Using external oscillatory technique, compliance measurement is obtained with each external oscillation cycle, but to measure systolic and diastolic pressures, compliance has to be measured over more than one cardiac cycle. In the presence of significant blood pressure variability due to respirations or arrhythmias, erroneous readings can be obtained.

Given bell-shaped arterial compliance dependence on the transmural pressure, multiple measurements with different Pe values have to be done to determine peak compliance, while at the same time arterial pressure changes resulting in multiple peaks.

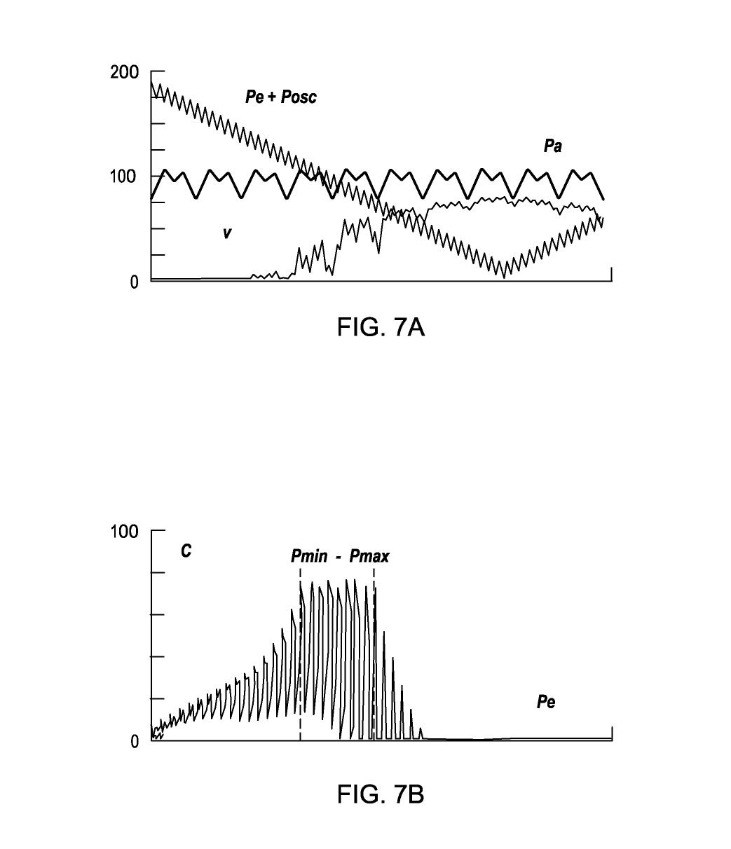

Reconstruction of arterial pressure waveform requires not only instantaneous measurement of compliance, but also knowing how compliance changes with the cuff pressure (dCa/dPe). If Pe changes slowly, like in FIG. 7C, arterial oscillation exceeds cuff pressure change and dCa/dPe will not be accurate, unless averaged over long time interval.

To measure dCa/dPe, changes in Pe have to be introduced as oscillation, i.e., pressure changes due to external oscillation and pulse pressure. External oscillation frequency is substantially higher than arterial pulse, and oscillation amplitude exceeds baseline change between two successive oscillations.

Applying external oscillation of known amplitude dVc_e_slow, can be used to calibrate the plethysmographic cuff (estimate cuff volume Vc_{—}1 at the beginning of oscillation:

Vc_{—}1=−(dVc_{—}e_slow/dPe)*Pe2.

### (Dubois A B, 1955 and Coates A L, 1997).

Changes of Vc out of phase of external oscillation are due to arterial volume changes.

Similarly, using higher frequency oscillation cuff volume can be calculated for each oscillation cycle:

Vc_{—}1=−(dVc_{—}e_fast/dPe)*Pe2.

If cuff air volume stays the same during measurement cycle, any variation in Vc at the same phase of external oscillation is due to blood volume variation Va(t). As seen below, baseline changes little between two successive oscillations.

Expanded segment of fast oscillation (0.2 s long). High frequency oscillation calculates compliance Ca(t). To know if the arterial pressure is above the Pe or below Pe, we can calculate dCa/dPe. If dCa/dPe>0, then Pa>Pe (increasing Pe compliance increases, decreasing-decreases. If dCa/dPe<0, then Pa<Pe (increasing Pe compliance decreases; decreasing-increases).

By superimposing low and high frequency oscillations, arterial compliance can be calculated for each high frequency oscillation pulse: dVa/dPe_fast and for each change in baseline between two successive oscillations: d(dVa/dPe_fast)/dPe.

Thus, relationships dVa/dPe(Pe) and Ca(Pe) are obtained and used to reconstruct Ca(Ptm); reverse function (Ptm(Ca) and Ca(t). Then arterial waveform can be reconstructed:

Pa(t)=Pe(t)+Ptm(Ca(t)).

Reconstructing of the arterial waveform is carried out with time resolution equal to one external oscillation period.

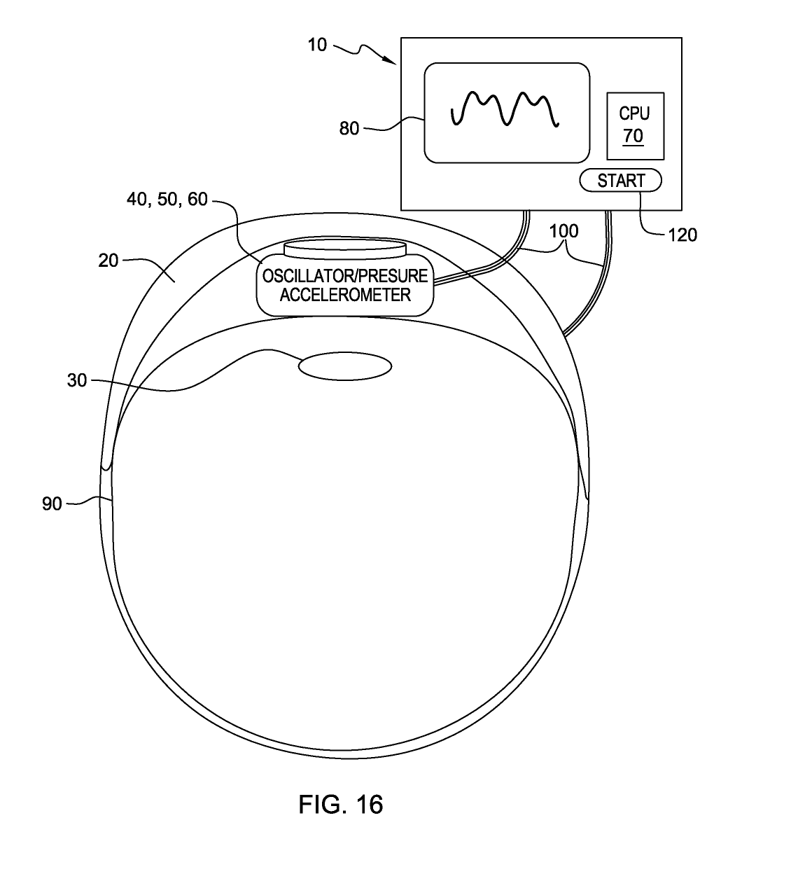

Embodiment with the vibrator induced forced oscillation may utilize a combined vibrator, accelerometer and pressure sensor 40, 50, 60 under the cuff above the artery (such as depicted in FIG. 16). Such a combined vibrator, accelerometer and pressure sensor under the cuff can be used to generate high frequency oscillation, measure its amplitude, sense cuff pressure and estimate Ca(Pe).

To eliminate venous congestion artifact distal occlusion cuff 15 has to be inflated or the combined vibrator, accelerometer and pressure sensor (FIG. 16) has to be placed close to the cuff edge proximal to the heart, so center and distal part of the cuff compresses veins in the arm. Without venous compression, arterial compliance can not be measured and volume clamp or external oscillation methods are invalid.

Pressure sensor, like piezoresistive or strain gauge element in this combined sensor senses cuff pressure Pe. Oscillator (vibratory motor) introduces forced oscillation and accelerometer registers amplitude of the induced oscillation. Amplitude and phase of the forced oscillation depends on the sensor mass and impedance of the partially compressed blood vessel and surrounding tissue. Since sensor mass and tissue properties does not change with external pressure Pe, oscillation amplitude will depend on arterial compliance Ca which is maximal at zero transmural pressure.

Thus, A(Pe) will depend on the transmural pressure, and will be maximum at the moments when Ptm=0. Changing cuff pressure Pe relationship A(Pe) will follow relationship (Ca(Pe)).

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

### LIST OF REFERENCES

Chen C H, Nevo E, Fetics B, Pak P H, Yin F C, Maughan W L, et al. Estimation of central aortic pressure waveform by mathematical transformation of radial tonometry pressure. validation of generalized transfer function. Circulation. 1997 Apr. 1; 95(7):1827-36.
Cloud G C, Rajkumar C, Kooner J, Cooke J, Bulpitt C J. Estimation of central aortic pressure by SphygmoCor requires intra-arterial peripheral pressures. Clin Sci (Lond). 2003 August; 105(2):219-25.
De Hert S G, Vermeyen K M, Moens M M, Hoffmann V L, Bataillie K J. Central-to-peripheral arterial pressure gradient during cardiopulmonary bypass: Relation to pre- and intra-operative data and effects of vasoactive agents. Acta Anaesthesiol Scand. 1994 July; 38(5):479-85.
Gravlee G P, Wong A B, Adkins T G, Case L D, Pauca A L. A comparison of radial, brachial, and aortic pressures after cardiopulmonary bypass. J Cardiothorac Anesth. 1989 February; 3(1):20-6.
Haluska B A, Jeffriess L, Mottram P M, Carlier S G, Marwick T H. A new technique for assessing arterial pressure wave forms and central pressure with tissue Doppler. Cardiovasc Ultrasound. 2007 Jan. 31; 5:6.
Hulsen H T, Nijdam M E, Bos W J, Uiterwaal C S, Oren A, Grobbee D E, et al. Spurious systolic hypertension in young adults; prevalence of high brachial systolic blood pressure and low central pressure and its determinants. J Hypertens. 2006 June; 24(6):1027-32.
Katsuno M. Usefulness of brachial artery pressure with forearm compression as arterial pressure monitoring after cardiopulmonary bypass. Masui. 1996 January; 45(1):77-81.
Melenovsky V, Borlaug B A, Fetics B, Kessler K, Shively L, Kass D A. Estimation of central pressure augmentation using automated radial artery tonometry. J Hypertens. 2007 July; 25(7):1403-9.
Pauca A L, Wallenhaupt S L, Kon N D. Reliability of the radial arterial pressure during anesthesia. is wrist compression a possible diagnostic test? Chest. 1994 January; 105(1):69-75.
Penaz J, Honzikova N, Jurak P. Vibration plethysmography: a method for studying the visco-elastic properties of finger arteries. Med Biol Eng Comput. 1997 November; 35(6):633-7.
Roman M J, Devereux R B, Kizer J R, Lee E T, Galloway J M, Ali T, et al. Central pressure more strongly relates to vascular disease and outcome than does brachial pressure: The strong heart study. Hypertension. 2007 July; 50(1):197-203.
Rulf E N, Mitchell M M, Prakash O, Rijsterborg H, Cruz E, Deryck Y L, et al. Measurement of arterial pressure after cardiopulmonary bypass with long radial artery catheters. J Cardiothorac Anesth. 1990 February; 4(1):19-24.
Sugimachi M, Shishido T, Miyatake K, Sunagawa K. A new model-based method of reconstructing central aortic pressure from peripheral arterial pressure. Jpn J Physiol. 2001 April; 51(2):217-22.
Thrush D N, Steighner M L, Rasanen J, Vijayanagar R. Blood pressure after cardiopulmonary bypass: Which technique is accurate? J Cardiothorac Vasc Anesth. 1994 June; 8(3):269-72.
Van Beck J O, White R D, Abenstein J P, Mullany C J, Orszulak T A. Comparison of axillary artery or brachial artery pressure with aortic pressure after cardiopulmonary bypass using a long radial artery catheter. J Cardiothorac Vasc Anesth. 1993 June; 7(3):312-5.
Wilkinson I B, Franklin S S, Hall I R, Tyrrell S, Cockcroft J R. Pressure amplification explains why pulse pressure is unrelated to risk in young subjects. Hypertension. 2001 Dec. 1; 38(6):1461-6.
Penaz J, Honzikova N, Jurak P. Vibration plethysmography: a method for studying the visco-elastic properties of finger arteries. Med Biol Eng Comput. 1997 November; 35(6):633-7.

What is claimed is:

1. A non-invasive method for measuring central arterial blood pressure in a patient under test, comprising the steps off:
fixing an inflatable cuff to the patient at a measurement site;
occluding blood flow distal the inflatable cuff to eliminate flow related gradient and to eliminate venous artifact from the distal venous stasis;
applying a variable external pressure to the measurement site using the inflatable cuff;
registering blood vessel or tissue containing blood vessel response to the applied variable external pressure (blood volume in the vessel or parameter that reflects blood volume in the vessel);
detecting points of maximal blood vessel response which coincide with maximal arterial compliance at zero transmural pressure; and
determining blood pressure in the measurement site from the steps of applying, registering, and detecting.

2. The non-invasive method for measuring central arterial blood pressure as set forth in claim 1, wherein the step of applying includes applying a slowly changing extrinsic pressure over time to realize a slowly changing transmural pressure over time.

3. The non-invasive method for measuring central arterial blood pressure as set forth in claim 1, wherein the step of applying includes applying a first oscillatory pressure signal at a frequency that is greater that the heartbeat.

4. The non-invasive method for measuring central arterial blood pressure as set forth in claim 3, wherein the step of applying further includes applying a second oscillatory pressure signal at a frequency that is at least twice that of the first oscillatory pressure signal.

5. The non-invasive method for measuring central arterial blood pressure as set forth in claim 3, wherein the step of determining includes calculating transmural pressure at each peak of the second oscillatory pressure signal.

6. The non-invasive method for measuring central arterial blood pressure as set forth in claim 1, wherein the central arterial blood pressure is equal to the extrinsic pressure minus the transmural pressure, wherein maximum compliance is at 0 TM pressure and wherein maximum volume change for the same pressure change is maximum compliance.

7. The non-invasive method for measuring central arterial blood pressure as set forth in claim 1, wherein the step of registering blood vessel or tissue containing blood vessel response to the applied variable external pressure includes detecting blood volume or detecting a parameter that reflects blood volume.

8. A method of measuring central blood pressure as recited in claim 1, wherein the step of registering includes using the inflatable cuff to detect cuff pressure oscillation and/or cuff pressure compliance.

9. A method of measuring central blood pressure as recited in claim 1, wherein the step of registering includes registering different forms of plethysmogram (photo, impedance, strain-gauge).

10. A method of measuring central blood pressure as recited in claim 1, wherein the step of registering includes registering vessel diameter and/or wall motion.

11. An apparatus for measuring a patient's central blood pressure, comprising
an inflatable cuff for applying and measuring pressure at a patient measurement site;
an occlusion device for occluding an artery and/or the artery's branches distal to the measurement site to eliminate flow related gradient and to eliminate venous artifact from the distal venous stasis;
a device for applying variable external pressure via the cuff at the patient measurement site;
a processor for processing data associated with a blood vessel or tissue containing blood vessel response to the applied variable external pressure, including the detected points of maximal blood vessel response that coincide with maximal arterial compliance at zero transmural pressure and determining blood pressure at the measurement site based on the blood vessel response data.

12. The apparatus as set forth in claim 11, wherein the occlusion device eliminates flow-related gradient and a pressure contribution from veins at the measurement site.

13. The apparatus as set forth in claim 11, wherein the device applies a first oscillatory pressure signal to a slowly changing extrinsic pressure signal over time, the first oscillatory pressure signal equal to or greater than the 60 cycles/second.

14. The apparatus as set forth in claim 13, wherein the device applies a second oscillatory pressure signal to a slowly changing extrinsic pressure signal over time, the second oscillatory pressure signal equal to or greater than twice the frequency of the first oscillatory pressure signal.

15. The apparatus as set forth in claim 13, wherein the processor determines the transmural pressure at each peak of the second oscillatory pressure signal.

16. The apparatus as set forth in claim 15, wherein the processor registers blood vessel or tissue containing blood vessel response to the applied variable external pressure based in a detected blood volume or parameter that reflects blood volume.

17. Method to measure central blood pressure noninvasively in a patient, comprising the steps of:
attaching an inflatable cuff to a measurement site on the patient's arm;
occluding blood flow distal the inflatable cuff using an occlusion cuff positioned on the arm distal the place of attachment of the inflatable cuff to minimize pressure gradient between the patient's central circulation and the brachial artery at the place of attachment of the inflatable cuff;
measuring the brachial pressure at the measurement site using any of a group of techniques consisting of tonometry, oscillometry with specific pre-occlusion calibration, active oscillometry and plethysmography with use of a volume clamp; and
calculating the arterial pressure at the measurement based on the brachial pressure at maximum compliance.
